(19) United States
(12) Patent Application Publication
(10) Pub. No.: US 2026/0356784 A1
(43) Pub. Date: Nov. 12, 2026

(54) CONVERSION OF A BIOMASS-DERIVED HYDROCARBON FEEDSTOCK TO PRODUCE ADIPIC ACID

(71) Applicant: IFP ENERGIES NOUVELLES, RUEIL-MALMAISON (FR)

(72) Inventors: Catherine LAROCHE, RUEIL-MALMAISON CEDEX (FR); Ludovic CHAHEN, RUEIL-MALMAISON CEDEX (FR)

(73) Assignee: IFP ENERGIES NOUVELLES, RUEIL-MALMAISON (FR)

(21) Appl. No.: 19/139,858

(22) PCT Filed: Dec. 12, 2023
(86) PCT No.: PCT/EP2023/085336

§ 371 Date: Jun. 16, 2025

(30) Foreign Application Priority Data
Dec. 20, 2022 (FR) 2213916

(51) Int. Cl. C07C 51/15; B01J 8/04; C07C 1/207; C12M 1/40; C12P 7/10

(52) U.S. Cl. CPC C07C 51/15; B01J 8/0492; C07C 1/2072; C12M 21/18; C12P 7/10; C12P 2201/00

(57) ABSTRACT

Disclosed is a method for converting biomass into adipic acid, successively involving:
a) a step of treating the biomass to produce ethanol and CO₂;
b) a step of converting the ethanol obtained at the end of step a) to obtain 1,3-butadiene and dihydrogen;
c) a step of synthesizing adipic acid from the 1,3-butadiene and the dihydrogen obtained at the end of step b) and from the CO₂ obtained at the end of step a).

### TECHNICAL FIELD

The invention relates to a process for treating biomass, preferably lignocellulosic biomass, to produce adipic acid from a single biobased carbon source while maximizing the carbon yield.

The process for fermenting lignocellulosic biomass makes it possible to produce ethanol and carbon dioxide (CO₂), which can be advantageously converted, compared to the prior art, into adipic acid.

### PRIOR ART

Adipic acid is a chemical compound used as a raw material as an intermediate compound, in particular for the synthesis of polyamides, polyesters or polyurethanes.

To date, several processes for producing adipic acid are known and used in industry. The main process used in industry consists in oxidizing cyclohexane using oxygen, to give the intermediate compounds cyclohexanol and cyclohexanone, generally a mixture of these two compounds. In a subsequent step, the intermediate compounds, cyclohexanol and cyclohexanone, are oxidized by nitric acid, in the presence of a catalyst, to give adipic acid.

Patent U.S. Pat. No. 5,487,987 describes a route for the synthesis of adipic acid from biomass, to form catechol first of all, which is then converted into cis, cis-muconic acid which can undergo reduction to form adipic acid.

Patent application US2011/0172475 describes a route for the synthesis of adipic acid, going via the production of isobutanol from biomass. The isobutanol is subsequently converted into 1,3-butadiene which is in turn converted into adiponitrile, then finally into adipic acid. 1,3-butadiene is a product of strategic importance for the synthesis of adipic acid. This compound can be obtained by dehydrating bioethanol. This type of process is disclosed in particular in applications WO21193457 and WO21006252, or in application WO21052968. It is well known that bioethanol can be produced by fermentation of sugars originating from various biomasses. However, fermentations to produce ethanol do not generally have good “carbon” yields, because some of this carbon is lost in the form of CO₂. Moreover, ethanol produced from “1st generation” (1G) biomass faces competition from the food-processing industry, and losing a large proportion of this resource in the form of a greenhouse gas is even more problematic.

The aim of the invention is to overcome all of the abovementioned disadvantages. More specifically, the aim of the invention is to develop a process for treating “second generation” (2G) lignocellulosic biomass to produce adipic acid from ethanol and CO₂ originating from fermentation.

The aim of the invention is to overcome all of the abovementioned disadvantages. More specifically, the aim of the invention is to develop a process for treating biomass, preferably “second generation” (2G) lignocellulosic biomass, to produce adipic acid from ethanol and CO₂ which originate from fermentation.

### SUBJECT MATTERS OF THE INVENTION

In the context described previously, a first object of the present description is to overcome the problems of the prior art and to upgrade carbon, and in particular biobased carbon in the form of CO₂, into high value-added compounds, and in particular into adipic acid. Specifically, the present invention relates to a process for producing adipic acid following an arrangement of steps, making it possible to convert the biomass into ethanol and CO₂ then to convert these products into adipic acid, using one or more of the following steps in addition to, or as a replacement for, some of the conventional steps for the synthesis of adipic acid.

According to a first aspect, the present invention relates to a process for converting biomass, preferably lignocellulosic biomass, into adipic acid, successively comprising:
a) a step of treating the biomass to produce ethanol and CO₂;
b) a step of converting the ethanol obtained on conclusion of step a), to obtain 1,3-butadiene;
c) a step of synthesizing adipic acid from the 1,3-butadiene obtained on conclusion of step b) and the CO₂ obtained on conclusion of step a).

The invention is based on upgrading CO₂, which is a by-product formed during the step of fermenting the biomass to give ethanol, and optionally reusing the water produced during the dehydration step in the other steps of the process according to the invention. This invention therefore has a sequence of individual operations which serve to maximize the carbon yield of the synthesis of adipic acid from biomass, preferably lignocellulosic biomass, and even more preferentially “second generation” (2G) lignocellulosic biomass.

According to one or more embodiments, step a) comprises the following substeps:
a1) a step of pretreating the biomass to obtain a pretreated substrate;
a2) a step of enzymatic or chemical hydrolysis of the pretreated substrate obtained on conclusion of step a1) to obtain an enzymatic or chemical hydrolysis must;
a3) a step of alcoholic fermentation of the enzymatic or chemical hydrolysis must obtained on conclusion of step a2) to obtain ethanol and CO₂.

According to one or more embodiments, substep a1) is carried out by steam explosion under acid conditions at a temperature of between 150 °C and 250 °C and for a period of between 5 minutes and 30 minutes.

According to one or more embodiments, substep a2) is carried out by enzymatic hydrolysis in the presence of Trichoderma reesei cellulases.

According to one or more embodiments, step b) comprises the following substeps:
b1) a step of converting the ethanol into acetaldehyde, comprising at least a reaction section fed at least with a fraction of the ethanol-rich effluent resulting from step b5), operated at a pressure of between 0.1 MPa and 1.0 MPa and at a temperature of between 200 °C and 500 °C in the presence of a catalyst, and a separation section making it possible to separate the effluent of said reaction section into at least a gaseous hydrogen effluent and a liquid ethanol/acetaldehyde effluent;
b2) a step of converting into butadiene, comprising at least a reaction section fed at least with a fraction of said ethanol/acetaldehyde effluent resulting from step b1), with a liquid ethanol-rich effluent resulting from step b3), with a fraction of the acetaldehyde-rich effluent resulting from step b5), operated in the presence of a catalyst at a temperature of between 300 °C and 400 °C and at a pressure of between 0.1 and 1.0 MPa, the feed flow rates being adjusted so that the ethanol/acetaldehyde molar ratio at the inlet of said reaction section is between 1 and 5, and a separation section making it possible to separate the effluent of said reaction section into at least a gaseous effluent and a liquid effluent;
b3) a step of treating the hydrogen, comprising at least a compression section compressing said hydrogen effluent resulting from step b1) to a pressure of between 0.1 MPa and 1.0 MPa and a gas/liquid scrubbing section fed at a temperature of between 15 °C and −30 °C with a fraction of said ethanol-rich effluent resulting from step b5) and with a fraction of said ethanol/acetaldehyde effluent resulting from step b1) and fed at a temperature of between 25 °C and 60 °C with said compressed hydrogen effluent, and producing at least a liquid ethanol-rich effluent and a purified hydrogen effluent;
b4) a step of extracting the butadiene, comprising at least a compression section compressing said gaseous effluent resulting from step b2) to a pressure of between 0.1 MPa and 1.0 MPa, a gas/liquid scrubbing section comprising a scrubbing column fed at the top, at a temperature of between 20 °C and −20 °C, with an ethanol stream consisting of said ethanol feedstock from the process and/or a fraction of the ethanol effluent resulting from step b5) and at the bottom with said gaseous effluent resulting from step b2), which is cooled, and a distillation section operated at a pressure of between 0.1 MPa and 1 MPa, fed at least with the liquid effluent resulting from said step b2) and with the liquid effluent from said gas/liquid scrubbing section, said step b4) producing at least one gaseous by-products effluent, a crude butadiene effluent and an ethanol/acetaldehyde/water effluent;
b4′) a step of first purification of the butadiene, comprising at least a gas/liquid scrubbing section fed at the bottom with the crude butadiene effluent resulting from b4) and at the top with a stream of water which may be a stream of water which originates outside said process for producing butadiene and/or a fraction of the water effluent resulting from step b5), said scrubbing section producing a pre-purified butadiene effluent at the top and a waste water effluent at the bottom;
b4″) a subsequent step of purifying the butadiene, which is fed at least with said pre-purified butadiene effluent resulting from said step b4′) and producing at least a purified butadiene effluent;
b5) a step of treating the effluents, which is fed at least with the water/ethanol/acetaldehyde raffinate resulting from step b5′), and producing at least an ethanol-rich effluent, an acetaldehyde-rich effluent and a water-rich effluent;
b5′) a step of removing impurities and brown oils, which is fed at least with the ethanol/acetaldehyde/water effluent resulting from step b4) and with the water-rich effluent resulting from step b5), and producing at least a water/ethanol/acetaldehyde raffinate, a light brown oils effluent and a heavy brown oils effluent;
b6) a step of washing with water, which is fed with the gaseous by-products effluent resulting from step b4) and also with a fraction of the water-rich effluent resulting from said step b5), producing at least one alcoholic water effluent.

According to one or more embodiments, step c) comprises the following substeps:
c1) a step of C—C coupling between two molecules of CO₂ and one molecule of 1,3-butadiene;
c2) a step of separating out hexenedioic acids;
c3) a step of reducing the hexenedioic acids formed in step c1) to give adipic acid.

According to one or more embodiments, substep c1) is carried out in the presence of a metal precursor, a ligand and a reducing agent in a solvent, characterized in that:
the metal precursor is a nickel (II) salt;
the ligand is a bidentate dinitrogen ligand;
the reducing agent is a metal selected from zinc or manganese;
the solvent is a polar aprotic solvent.

According to one or more embodiments, substep c1) is carried out at a temperature of between 5 °C and 70 °C.

According to one or more embodiments, substep c3) is carried out in the presence of a catalyst and a reducing agent, said catalyst being palladium on activated carbon, and the reducing agent being the purified hydrogen originating from substep b3).

According to one or more embodiments, the biomass is a lignocellulosic biomass.

According to a second aspect, the present invention relates to a plant suitable for implementing the process according to the invention, said plant comprising:
a first reaction section making it possible to produce ethanol and CO₂ from biomass;
a second reaction section for converting the ethanol into 1,3-butadiene and dihydrogen; and
a third reaction section making it possible to produce adipic acid from 1,3-butadiene, CO₂ and dihydrogen.

### LIST OF FIGURES

FIG. 1 shows a schematic depiction of the process and of the plant according to the present invention, making it possible to produce adipic acid from biomass, preferably lignocellulosic biomass.

### DESCRIPTION OF THE EMBODIMENTS

Embodiments of the process according to the first aspect of the invention and of the plant according to the second aspect of the invention will now be described in detail. In the following detailed description, many specific details are disclosed in order to provide a deeper understanding of the process and of the plant. However, it will be apparent to those skilled in the art that the process and the plant can be implemented without these specific details. In other cases, well-known features have not been described in detail in order to avoid unnecessarily complicating the description.

In the present patent application, the term “to comprise” is synonymous with (means the same thing as) “to include” and “to contain”, and is inclusive or open and does not exclude other elements which are not stated. It is understood that the term “to comprise” includes the exclusive and closed term “to consist of”. Moreover, in the present description, an effluent comprising essentially or solely compounds A corresponds to an effluent comprising at least 90% by weight, preferably at least 95% by weight, very preferably at least 99% by weight, of compounds A.

In the present application, the groups of chemical elements are given, by default, according to the CAS classification (CRC Handbook of Chemistry and Physics, published by CRC Press, Editor-in-Chief D. R. Lide, 81st edition, 2000-2001). For example, group VIII (or group VIIIB) according to the CAS classification corresponds to the metals from columns 8, 9 and 10 according to the new IUPAC classification; group VIB according to the CAS classification corresponds to the metals from column 6 according to the new IUPAC classification.

In the present application, biomass means any feedstock produced biologically, preferably by fermentation of sugars originating, for example, from sugar-producing plant crops, such as sugar cane (saccharose, glucose, fructose and sucrose), from beetroot, or from starchy plants (starch) or from lignocellulosic biomass or from hydrolyzed cellulose (predominantly glucose, and xylose, galactose), containing variable amounts of water. The biomass is preferably lignocellulosic biomass, and even more preferentially “second generation” (2G) lignocellulosic biomass.

The present invention can be defined as a process comprising a sequence of reaction steps making it possible to produce adipic acid from biomass, preferably lignocellulosic biomass, and even more preferentially “second generation” (2G) lignocellulosic biomass. More particularly, the present invention relates to a process for converting biomass into adipic acid, successively comprising:
a) a step of treating the biomass to produce ethanol and CO₂;
b) a step of converting the ethanol obtained on conclusion of step a), to obtain 1,3-butadiene;
c) a step of synthesizing adipic acid from the 1,3-butadiene obtained on conclusion of step b) and the CO₂ obtained on conclusion of step a).

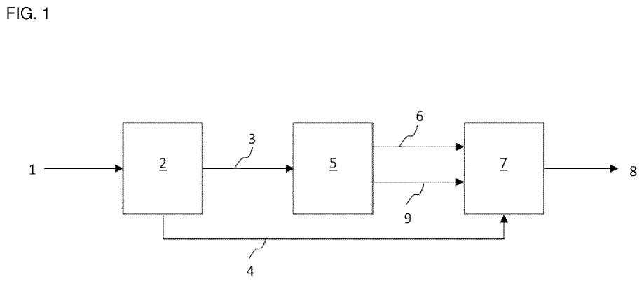

Moreover, the present invention can also be defined as a plant which is able to implement the process according to the invention, as illustrated in FIG. 1, said plant comprising, in particular:
a first reaction section 2 making it possible to produce ethanol 3 and CO₂ 4 from biomass 1, preferably lignocellulosic biomass;
a second reaction section 5 for converting the ethanol 3 into 1,3-butadiene 6 and hydrogen 9; and
a third reaction section 7 making it possible to produce adipic acid 8 from 1,3-butadiene 6, hydrogen 9 and CO₂ 4.

### First Reaction Section (Step a) of the Process According to the Invention)

The first reaction section 2 makes it possible to produce ethanol 3 and CO₂ 4 from biomass 1.

In an embodiment according to the invention, the biomass used in the process is lignocellulosic biomass, preferably “second generation” lignocellulosic biomass. Wood from hardwood trees and cereal straw are the most commonly used substrates. They predominantly consist of approximately 40% to 50% of cellulose, 20% to 25% of hemicellulose and 15% to 25% of lignin. Other resources, dedicated forestry crops, residues from plants used to produce alcohol, sugar and cereals, residues from the paper industry and products of the conversion of cellulosic and lignocellulosic materials can be used.

In one embodiment according to the invention, the process for converting biomass into ethanol more particularly comprises the following substeps:
a1) a step of pretreating the biomass to obtain a pretreated substrate;
a2) a step of enzymatic or chemical hydrolysis of the cellulosic residue obtained on conclusion of step a1) to obtain an enzymatic or chemical hydrolysis must;
a3) a step of alcoholic fermentation of the enzymatic or chemical hydrolysis must obtained on conclusion of step a2) to obtain ethanol and CO₂.
   Physicochemical Pretreatment (Step a1))

The pretreatment step a1) makes it possible to produce a pretreated substrate comprising the sugars contained in the hemicelluloses in the form of monomers, essentially pentoses, such as xylose and arabinose, and hexoses, such as galactose, mannose and glucose, and to improve the accessibility of the cellulose embedded in the lignin and hemicelluloses matrix. A wide range of technologies are available, including acid cooking, alkaline cooking, steam explosion and organosolv pulping treatments. The effectiveness of the pretreatment is measured by the hemicellulose recovery rate and by the susceptibility of the cellulose residue to hydrolysis. Acid pretreatments, under mild conditions, and by steam explosion, are the best suited as they allow total recovery of the pentoses and good accessibility of the cellulose to hydrolysis.

Preferentially, pretreatment step a1) is carried out by steam explosion under acid conditions at a temperature of advantageously between 150 °C and 250 °C and for a period of advantageously between 5 and 30 minutes. In this embodiment, step a1) allows the hemicelluloses to be converted into monomers while at the same time minimizing losses, in particular of furfural, xylose being the predominant sugar. The sugars released are then extracted by washing in the aqueous phase. The solid residue (i.e. the pretreated substrate) obtained on conclusion of the extraction then contains only cellulose and lignin.

Enzymatic or Chemical Hydrolysis (Step a2))

The pretreated substrate obtained on conclusion of step a1) is then hydrolysed, either acidically (i.e. chemically) or enzymatically using cellulolytic and/or hemicellulolytic enzymes. Microorganisms, such as fungi belonging to the genera Trichoderma, Aspergillus, Penicillium or Schizophyllum, or anaerobic bacteria belonging, for example, to the genus Clostridium, produce these enzymes, containing in particular cellulases and xylanases, suitable for the total hydrolysis of polymers constituting plants.

The acidic route, performed with strong acid, and more particularly with sulfuric acid, is effective but requires large amounts of chemical products (acid and then base for neutralization). Enzymatic hydrolysis does not have this drawback; moreover, it is performed under mild conditions and is effective.

In a preferential manner, the pretreated substrate, freed or not freed of the hydrolyzed hemicellulosic fraction and, where appropriate, the lignin, is hydrolyzed by the cellulolytic and/or hemicellulolytic enzymes produced by the specialized strains, Trichoderma reesei cellulases being the most effective and the most appropriate when the carbon-based substrates are derived from cellulosic or lignocellulosic biomass. The pretreated substrate to be hydrolyzed is preferably suspended in an aqueous phase in a proportion of 6 to 25% dry matter, preferably 10 to 20%, the pH is adjusted to between 4 and 5.5, preferably between 4.8 and 5.2 and the temperature to between 40 °C and 60 °C, preferably between 45 °C and 50 °C. The hydrolysis reaction is started by adding the cellulases; the amount usually used is from 10 mg to 30 mg of excreted proteins per gram of pretreated substrate. The reaction generally lasts from 15 hours to 48 hours depending on the efficacy of the pretreatment, the composition of the cellulase mixture and the amount of enzymes added. The reaction is monitored by assaying the sugars released, in particular glucose. The sugar solution (must) is then separated from the non-hydrolyzed solid fraction, essentially consisting of lignin, by filtration or centrifugation; this must is used for ethanol fermentation. When the cellulose fraction has been freed of the hydrolyzed hemicelluloses in the treatment step, glucose is the predominant sugar contained in the must.

Fermentation (Step a3))

Alcoholic fermentation is a biochemical process in which the sugars (carbohydrates, principally glucose) contained in the must are converted into alcohol, preferentially into ethanol, in a liquid medium deprived of air (anaerobic). The step of fermenting sugars to obtain ethanol is well known to those skilled in the art.

Alcoholic fermentation is preferentially performed at a temperature of between 25 °C and 32 °C. For a more complete description of the conventional fermentation processes, reference may be made to the textbook “Les Biocarburants, État des lieux, perspectives et enjeux du développement [Biofuels, current situation, perspectives and development challenges]”, Daniel Ballerini, published by Technip, 2006.

In general, ethanol is separated from the fermentation must by distillation and the residue consists of the distillation stillage. Regular or continuous distillation of the ethanol is necessary because, above 14% ethanol, certain yeasts may be “poisoned”, resulting in a loss of productivity. Distillation is performed to make it possible to produce an ethanol feedstock suitable for the dehydration process described later.

The CO₂ is recovered in the form of gas at the fermenter outlet. According to an essential aspect of the invention, the CO₂ obtained on conclusion of the fermentation step is sent at least partly to the adipic acid synthesis step (step c) of the process according to the invention). The CO₂ may be compressed before storage or before use by means of compressors.

The ethanol fermentation residue, after separation from the ethanol, may be used as an inductive carbon source or as the main carbon source for enzyme production. The concentration of this residue is preferably adjusted to obtain the carbon source concentration best suited for the cellulolytic and/or hemicellulolytic enzyme production process.

The enzymatic hydrolysis and fermentation steps may be performed simultaneously (Simultaneous Saccharification and Fermentation (SSF) process), and then advantageously followed by a step of distillation and separation of the alcohol obtained.

### Second Reaction Section (Step b) of the Process According to the Invention)

The second reaction section 5 makes it possible to produce 1,3-butadiene 6 from the ethanol 3 resulting from the first reaction section 2. There are a number of processes in the literature making it possible to obtain 1,3-butadiene from ethanol, in particular the process disclosed in patent application FR3026100 which is described herein by way of example.

Advantageously, the ethanol feedstock used in step b) of the process according to the invention is a feedstock comprising at least 80% by weight of ethanol relative to the total weight of the feedstock, preferentially at least 90% by weight, and preferably at least 93% by weight. Very preferably, said ethanol feedstock meets the EN 15376 fuel ethanol specifications.

In one embodiment according to the invention, step b) of the process according to the invention comprises the following substeps:
b1) a step of converting the ethanol into acetaldehyde, comprising at least a reaction section fed at least with a fraction of the ethanol-rich effluent resulting from step b5), operated at a pressure of between 0.1 MPa and 1.0 MPa and at a temperature of between 200 °C and 500 °C in the presence of a catalyst, and a separation section making it possible to separate the effluent of said reaction section into at least a gaseous hydrogen effluent and a liquid ethanol/acetaldehyde effluent;
b2) a step of converting into butadiene, comprising at least a reaction section fed at least with a fraction of said ethanol/acetaldehyde effluent resulting from step b1), with a liquid ethanol-rich effluent resulting from step b3), with a fraction of the acetaldehyde-rich effluent resulting from step b5), operated in the presence of a catalyst at a temperature of between 300 °C and 400 °C and at a pressure of between 0.1 and 1.0 MPa, the feed flow rates being adjusted so that the ethanol/acetaldehyde molar ratio at the inlet of said reaction section is between 1 and 5, and a separation section making it possible to separate the effluent of said reaction section into at least a gaseous effluent and a liquid effluent;
b3) a step of treating the hydrogen, comprising at least a compression section compressing said hydrogen effluent resulting from step b1) to a pressure of between 0.1 MPa and 1.0 MPa and a gas/liquid scrubbing section fed at a temperature of between 15 °C and −30 °C with a fraction of said ethanol-rich effluent resulting from step b5) and with a fraction of said ethanol/acetaldehyde effluent resulting from step b1) and fed at a temperature of between 25 °C and 60 °C with said compressed hydrogen effluent, and producing at least a liquid ethanol-rich effluent and a purified hydrogen effluent;
b4) a step of extracting the butadiene, comprising at least a compression section compressing said gaseous effluent resulting from step b2) to a pressure of between 0.1 MPa and 1.0 MPa, a gas/liquid scrubbing section comprising a scrubbing column fed at the top, at a temperature of between 20 °C and −20 °C, with an ethanol stream consisting of said ethanol feedstock from the process and/or a fraction of the ethanol effluent resulting from step b5) and at the bottom with said gaseous effluent resulting from step b2), which is cooled, and a distillation section operated at a pressure of between 0.1 MPa and 1 MPa, fed at least with the liquid effluent resulting from said step b2) and with the liquid effluent from said gas/liquid scrubbing section, said step b4) producing at least one gaseous by-products effluent, a crude butadiene effluent and an ethanol/acetaldehyde/water effluent;
b4′) a step of first purification of the butadiene, comprising at least a gas/liquid scrubbing section fed at the bottom with the crude butadiene effluent resulting from b4) and at the top with a stream of water which may be a stream of water which originates outside said process for producing butadiene and/or a fraction of the water effluent resulting from step b5), said scrubbing section producing a pre-purified butadiene effluent at the top and a waste water effluent at the bottom;
b4″) a subsequent step of purifying the butadiene, which is fed at least with said pre-purified butadiene effluent resulting from said step b4′) and producing at least a purified butadiene effluent;
b5) a step of treating the effluents, which is fed at least with the water/ethanol/acetaldehyde raffinate resulting from step b5′), and producing at least an ethanol-rich effluent, an acetaldehyde-rich effluent and a water-rich effluent;
b5′) a step of removing impurities and brown oils, which is fed at least with the ethanol/acetaldehyde/water effluent resulting from step b4) and with the water-rich effluent resulting from step b5), and producing at least a water/ethanol/acetaldehyde raffinate, a light brown oils effluent and a heavy brown oils effluent;
b6) a step of washing with water, which is fed with the gaseous by-products effluent resulting from step b4) and also with a fraction of the water-rich effluent resulting from said step b5), producing at least one alcoholic water effluent.

Steps b1) to b6) are described in detail below.

Step b1) of Converting the Ethanol into Acetaldehyde

In accordance with one embodiment according to the invention, a step b1) of converting the ethanol into acetaldehyde comprises at least a reaction section fed at least with a fraction of the ethanol-rich effluent resulting from step b5), said fraction preferably constituting at least 10% of the flow of said ethanol-rich effluent resulting from step b5), and optionally advantageously fed with at least one fraction of said ethanol feedstock, and a separation section making it possible to separate the effluent of said reaction section into at least a gaseous hydrogen effluent and a liquid ethanol/acetaldehyde effluent.

Said reaction section makes it possible to convert the ethanol into acetaldehyde in the presence of a catalyst, preferably consisting of a mixture of chromium oxide and copper oxide, or any other suitable catalyst. These catalysts are well known to those skilled in the art.

Said reaction section is operated at a pressure of between 0.1 MPa and 1.0 MPa, preferably between 0.1 MPa and 0.5 MPa, preferably between 0.1 MPa and 0.3 MPa, and at a temperature of between 200 °C and 500 °C, preferentially of between 250 °C and 300 °C.

Preferably, the conversion of the ethanol is between 30 and 40%, with a selectivity of between 85 and 100% toward acetaldehyde, preferably between 90 and 95% toward acetaldehyde. The effluent of said reaction section also comprises by-products such as crotonaldehyde, butyraldehyde, diethyl acetal, ethyl acetate and acetic acid.

Said separation section uses gas/liquid separation means known to those skilled in the art. Use will preferably be made of a gas/liquid separator operated at a pressure of between 0.1 MPa and 0.3 MPa and at a temperature of between 25 °C and 60 °C.

Step b2) of Converting an Ethanol/Acetaldehyde Mixture into Butadiene

In accordance with one embodiment according to the invention, a step b2) of converting into butadiene comprises at least a reaction section fed at least with a fraction of said ethanol/acetaldehyde effluent resulting from step b1), with a liquid ethanol-rich effluent resulting from step b3), with an acetaldehyde-rich fraction of the effluent resulting from step b5) and optionally advantageously fed with an ethanol-rich stream resulting from step b5), and a separation section making it possible to separate the effluent of said reaction section into at least a gaseous effluent and a liquid effluent. Said reaction section can also be fed with an external stream of acetaldehyde.

The flow rate of the various feeds to the reaction section of said step b2) is adjusted such that ethanol/acetaldehyde molar ratio at the inlet of said reaction section is between 1 and 5, preferably between 1 and 3.5, even more preferably between 2 and 3, and very preferably between 2.4 and 2.7.

Said reaction section makes it possible to convert a portion of the ethanol/acetaldehyde mixture into at least butadiene. The conversion selectivity of the ethanol/acetaldehyde mixture is preferably greater than 60%, preferably greater than 70%, very preferably greater than 80%. Selectivity means the molar ratio of the flow rate of butadiene in the effluent from said reaction section to the flow rate of ethanol and acetaldehyde consumed in said reaction section. The conversion of the ethanol/acetaldehyde mixture is preferably greater than 30%, preferably greater than 40%, preferably greater than 47%. Conversion means the molar ratio of the flow rate of ethanol and acetaldehyde in the effluent from said reaction section to the flow rate of ethanol and acetaldehyde in the feed of said reaction section. It is operated in the presence of a catalyst, advantageously a silica-supported catalyst selected from the group consisting of catalysts comprising tantalum oxide, zirconium oxide or niobium oxide, preferentially comprising 2% tantalum oxide (see, for example, Corson, Jones, Welling, Hincbley, Stahly, Ind. Eng Chem. 1950, 42, 2, 359-373). Said second reaction zone is operated at a temperature of between 300 °C and 400 °C, preferably between 320 °C and 370 °C and at a pressure of between 0.1 MPa and 1.0 MPa, preferably between 0.1 MPa and 0.5 MPa, preferably between 0.1 MPa and 0.3 MPa.

Preferably, approximately 65 to 80% of the acetaldehyde is converted in said reaction section. The effluent from said reaction section therefore still comprises ethanol. Numerous impurities can be produced with the butadiene, including ethylene, propylene, diethyl ether (DEE), ethyl acetate, butanol, hexanol, butenes, pentenes, pentadienes, hexenes and hexadienes.

Since said reaction section is fed with the acetaldehyde effluent resulting from step b5) of treating the effluents, the ethanol to acetaldehyde ratio at the inlet to this section is adjusted by monitoring the fraction of the ethanol effluent resulting from said step b5) that feeds step b1) and therefore produces acetaldehyde. Indeed, the remaining fraction of the ethanol-rich effluent resulting from said step b5) feeds step b3) of treating the hydrogen and forms, after scrubbing the hydrogen effluent, said liquid ethanol-rich effluent resulting from said step b3), which feeds said step b2). This ethanol-rich liquid effluent resulting from said step b3) only contains a very small amount of acetaldehyde.

Said separation section uses gas/liquid separation means known to those skilled in the art. Use will preferably be made of a gas/liquid separator operated at a pressure of between 0.1 MPa and 0.3 MPa and at a temperature of between 25 °C and 60 °C.

### Step b3) of Treating the Hydrogen

In accordance with one embodiment, a step b3) of treating the hydrogen comprises at least a compression section fed with said hydrogen effluent resulting from step b1) and a gas/liquid scrubbing section fed with a fraction of said ethanol effluent resulting from step b5) and with a fraction of said ethanol/acetaldehyde effluent resulting from step b1), and produces at least a liquid ethanol-rich effluent and a purified hydrogen effluent. Preferably, said step b3) is not fed with any other streams.

Said fraction of said ethanol/acetaldehyde effluent resulting from step b1) is between 0 and 100%. The use of a fraction of said ethanol/acetaldehyde effluent makes it possible to decrease the flow rate of the fraction of said ethanol-rich effluent resulting from said step b5).

Said step b3) makes it possible to obtain a purified hydrogen effluent of high purity, i.e. comprising at least 90 mol % hydrogen, preferably 99 mol % hydrogen, preferably 99.8 mol % hydrogen. The purified hydrogen effluent also comprises traces of water and ethanol. This step also makes it possible to recover the ethanol and acetaldehyde included in the hydrogen effluent resulting from said step b1), thereby enabling them to be recycled and maximizing the overall yield of the process.

The use of a fraction of the ethanol-rich effluent resulting from step b5) and a fraction of the ethanol/acetaldehyde effluent resulting from step b1) instead of water—as was the practice in the prior art—makes it possible to decrease the total flow rate of water circulating in the process. Thus, the total flow rate of water feeding steps b5) and b5′) of treating effluents is decreased, thereby decreasing the size of the equipment and the consumption of utilities in steps b5) and b5′). Moreover, as described above, the ethanol-rich effluent resulting from said step b3) can directly feed step b2) for converting into butadiene, without having to be treated by step b5) of treating effluents.

The hydrogen effluent resulting from step b1) is compressed in a compression section to a pressure of between 0.1 MPa and 1.0 MPa, advantageously between 0.1 MPa and 0.7 MPa, and preferably between 0.4 MPa and 0.68 MPa. The effect of this compression is firstly to decrease the gas volume flow rate, and secondly to improve the efficacy of the downstream scrubbing.

The compressed hydrogen effluent is then cooled to a temperature of between 25 and 60 °C, preferentially between 30 °C and 40 °C, then feeds the bottom of the scrubbing column of the scrubbing section, in which it is brought into contact with said fraction of the ethanol effluent resulting from step b5) and said fraction of the ethanol/acetaldehyde effluent resulting from step b1), said fractions being fed, respectively, at the top and at an intermediate point in said scrubbing column. These two fractions are each cooled to a temperature of between 15 °C and −30 °C, preferentially between 0 °C and −15 °C, before being fed to said scrubbing column. Advantageously, the ethanol-rich effluent resulting from step b5) will be fed at a temperature below that of said fraction of the ethanol/acetaldehyde effluent resulting from step b1), thereby creating a thermal gradient between the top and the bottom and limiting losses of solvent in the purified hydrogen effluent. The gas/liquid scrubbing column of the scrubbing section is equipped with trays or random or structured packing.

Optional Step b3′) of Final Treatment of the Hydrogen Effluent

A step b3′) of final treatment of the hydrogen is advantageously carried out on conclusion of step b3). Said step b3′) comprises at least one gas/liquid scrubbing section fed with the purified hydrogen effluent resulting from b3), and with a pure water effluent originating from outside the process, or with a water-rich effluent resulting from step b5), and produces a purified hydrogen effluent and a waste water effluent.

Said scrubbing section comprises at least one gas/liquid scrubbing section fed at the bottom with said purified hydrogen effluent resulting from b3), and at the top with a pure water effluent or with a water-rich effluent resulting from step b5), and produces a purified hydrogen effluent at the top and a waste water effluent at the bottom.

This step makes it possible to recover the final traces of ethanol that may be contained in the purified hydrogen effluent resulting from b3). Said step b3′), which is similar to the hydrogen treatment according to the prior art, nevertheless employs water flow rates that are much lower than those used in the prior art since the hydrogen effluent feeding step b3′) has been treated beforehand by step b3) and therefore freed of all the acetaldehyde entrained in the hydrogen effluent resulting from step b1). With all other things being equal, the removal of traces of ethanol by washing with water requires lower flow rates than the removal of traces of acetaldehyde. Moreover, since ethanol is less volatile than acetaldehyde, it is entrained to a much lesser extent than acetaldehyde under the same operating conditions.

### Step b4) of Extracting the Butadiene

In accordance with one embodiment according to the invention, a step b4) of extracting the butadiene comprising at least a compression section, a gas/liquid scrubbing section and a distillation section is fed at least with said gaseous and liquid effluents resulting from said step b2), with an ethanol stream consisting of said ethanol feedstock of the process and/or a fraction of the ethanol effluent resulting from step b5), and produces at least one gaseous by-products effluent, a crude butadiene effluent and an ethanol/acetaldehyde/water effluent.

Said ethanol stream feeding step b4) preferably comprises at least 80% by weight of ethanol, preferentially at least 90% by weight, and preferably at least 93% by weight. Said ethanol stream feeding step b4) may contain methanol, water, ethyl acetate, butanol and hexanol. Preferably, said ethanol stream feeding step b4) preferably comprises less than 10% by weight of acetaldehyde, preferably less than 5% by weight, and preferably less than 1% by weight. Preferably, said ethanol stream feeding step b4) preferably comprises less than 20% by weight of water, preferably less than 5% by weight, preferably less than 1% by weight.

In a preferred arrangement, said ethanol stream feeding step b4) consists of said ethanol feedstock of the process. One advantage to this arrangement is that said feedstock is free of the reaction by-products that are formed in steps b1) and b2) and that can become concentrated due to the recycling. In particular, this ethanol feedstock does not contain acetaldehyde, or only contains trace amounts thereof.

In another preferred arrangement, said ethanol stream consists of a fraction of the ethanol effluent resulting from step b5) of treating the effluents.

The use of an ethanol stream containing very little, or no, acetaldehyde minimizes the entrainment of acetaldehyde into said gaseous by-products effluent withdrawn at the top of said gas/liquid scrubbing section, reducing the overall yield losses of the process and also the washing water flow rate required in step b6).

The gaseous effluent resulting from step b2) is compressed in said compression section to a pressure of between 0.1 MPa and 1.0 MPa, preferentially between 0.1 MPa and 0.7 MPa, and preferably between 0.2 MPa and 0.5 MPa. The effect of this compression is firstly to decrease the gas volume flow rate, and secondly to improve the efficacy of the downstream scrubbing. Preferably, the compressed gaseous effluent is then cooled to a temperature of between 25 °C and 60 °C, preferentially between 30 °C and 40 °C.

Preferably, said gas/liquid scrubbing section of step b4) comprises a scrubbing column fed at the top with said ethanol stream that feeds step b4) and at the bottom by said cooled compressed gaseous effluent, and produces the gaseous by-products effluent at the top and a liquid effluent at the bottom which feeds said distillation section of step b4).

Said ethanol stream feeding step b4) is cooled, before being fed to the top of said gas/liquid scrubbing column of the scrubbing section, to a temperature of between 20 °C and −20 °C, preferentially between 15 °C and 5 °C. The benefit of cooling said ethanol stream is an improvement in the performance of the scrubbing operation by minimizing the entrainment of ethanol and acetaldehyde in said gaseous by-products stream. Thus, all the butadiene present in the compressed and cooled gaseous effluent resulting from step b2) is done away with, and the vapor by-products effluent withdrawn at the top of said gas/liquid scrubbing section is devoid of butadiene.

Incidentally, minimizing the entrainment of acetaldehyde in said gaseous by-products effluent makes it possible to significantly decrease the flow rate of water required in step b6) of washing the gaseous by-products with water, the aim of which is to recover the ethanol and any traces of acetaldehyde entrained in the gaseous by-products effluent withdrawn at the top of the ethanol scrubbing section of step b4).

Preferably, the butadiene-enriched ethanol stream withdrawn at the bottom of said gas/liquid scrubbing section of step b4), and also the liquid effluent of step b2), feed said distillation section of step b4) so as to separate off at the top a vapor effluent comprising the majority of the butadiene, referred to as crude butadiene effluent, and at the bottom an ethanol/acetaldehyde/water residue. The term “the majority” means more than 80% of the butadiene included in the feed of said distillation section, preferentially more than 90%, preferably more than 95%, even more preferably more than 98%, very preferably more than 99% and very advantageously all of the butadiene included in said feed. This ethanol/acetaldehyde/water residue comprises ethanol and acetaldehyde, and also comprises the water produced in step b2) and the by-products formed in steps b1) and b2), for instance diethyl ether, ethyl acetate and brown oils. Said ethanol/acetaldehyde/water residue then feeds step b5′) of treating the effluents. Said distillation section is operated at a pressure of between 0.1 MPa and 1 MPa and preferably between 0.2 MPa and 0.5 MPa.

Step b4′) of First Purification of the Butadiene

Step b4′) of first purification of the butadiene comprises at least a gas/liquid scrubbing section fed at the bottom with the crude butadiene effluent resulting from b4) and at the top with a stream of water which may be a stream of water which originates outside said process for producing butadiene and/or a fraction of the water effluent resulting from step b5), said scrubbing section producing a pre-purified butadiene effluent at the top and a waste water effluent at the bottom. Preferably, said stream of water is a stream of water originating from outside the process.

Said waste water effluent contains acetaldehyde and a small amount of butadiene and can be sent to step b5) of treating the effluents, to the acetaldehyde distillation section, or to step b5′).

The aim of step b4′) is to remove the polar impurities, in particular the acetaldehyde which must not be present beyond a few ppm in the final butadiene. The crude butadiene effluent resulting from b4) comprises the majority of the butadiene, but still contains numerous impurities, including a large amount of acetaldehyde which forms an azeotrope with the butadiene and therefore cannot be completely removed by distillation during step b4). Thus, the flow rate of said stream of water is adjusted to obtain the desired specification for acetaldehyde in the pre-purified butadiene effluent.

Said stream of water is cooled to a temperature below 25 °C, preferably below 20 °C, before feeding the gas/liquid scrubbing section so as to perform the washing with a reduced amount of water. The feed temperature of said stream of water is chosen so as not to form hydrates with the butadiene and the light hydrocarbons still present in the butadiene stream resulting from step b4). The pressure of the scrubbing column is determined so as to ensure that there is no condensation of the butadiene and that it remains in gaseous form. The pressure in this step is between 0.1 MPa and 1 MPa and even more preferably between 0.2 MPa and 0.3 MPa.

Subsequent Step b4″) of Purification of the Butadiene

In accordance with one embodiment according to the invention, a subsequent step b4″) of purifying the butadiene is fed at least with said pre-purified butadiene effluent resulting from said step b4′) and produces at least a purified butadiene effluent.

This step b4″) makes it possible to purify the butadiene produced in the reaction steps to a very high purity level (more than 99.5% by weight, preferentially more than 99.8% by weight, and very preferentially more than 99.9% by weight) while limiting product losses by separating out the impurities which were not, or were only partially, withdrawn during step b4), b4′).

In a first embodiment of the invention, said step b4″) comprises at least one drying section, a cryogenic distillation section and a section for separating butadiene/butenes by liquid-liquid extraction.

The pre-purified butadiene effluent resulting from step b4′) feeds a drying section. The aim of this section is to achieve the required specifications for water in the final product (purified butadiene effluent) and to make it possible to carry out cryogenic separation without the risk of hydrates forming. A dry butadiene effluent is obtained at the outlet of said drying section. Dry butadiene means less than 10 ppm of water, preferably less than 5 ppm, preferably less than 1 ppm.

Said drying section preferentially comprises drying consisting of one or more volumes containing one or more adsorbents having a strong affinity for water. In a non-limiting manner, this adsorbent may consist of silica and/or alumina. In a non-limiting manner, this adsorbent may be a zeolite such as a zeolite 3A or 4A. When the adsorbent(s) are saturated with water, said pre-purified butadiene effluent is fed to another volume containing fresh or regenerated adsorbent(s).

The adsorbent can be regenerated by modifying the partial pressure of water within the volume, by modifying the temperature within the volume, or by modifying the partial pressure of water and the temperature within the volume. In the latter embodiment, the water-saturated adsorbent(s) are regenerated by heating the volume while feeding it with a stream that contains no water or very little water. “No water or very little water” means less than 500 ppm, preferentially less than 350 ppm, preferably less than 10 ppm, preferably less than 5 ppm, very preferably less than 1 ppm. This stream that contains no water or very little water can be, in a non-limiting manner, a nitrogen stream, an air stream, a hydrocarbon stream or a hydrogen stream. In a preferred embodiment of the invention, a fraction of the purified hydrogen effluent resulting from step b3) is used.

Said stream that contains no water or very little water is heated to a sufficient temperature to regenerate the adsorbent(s) before being fed to the volume containing the adsorbent(s) to be regenerated, preferentially to around 250 °C.

According to this first embodiment, said dry butadiene effluent then feeds a cryogenic distillation section that uses a distillation column. The light products leave at the top of the cryogenic distillation column at between −25 °C and −35 °C. The bottom of the column is at a temperature of between 20 °C and 50 °C, preferentially between 25 °C and 45 °C, very preferably between 30 °C and 40 °C, and the pressure at the top of the column is between 0.3 MPa and 0.4 MPa, preferentially 0.35 MPa. The benefit of the column is that of having very high efficacy in separating out the last incondensables, without losing butadiene (less than 0.05%). This thereby avoids significant recycling to step b4) and a loss of butadiene.

Still according to this first embodiment, the bottom product of said cryogenic distillation section, referred to as topped butadiene effluent, comprises butenes as main impurity. Said topped butadiene effluent feeds a section for separating butadiene/butenes by liquid-liquid extraction, as described in patent FR 2,036,057.

Said section for separating butadiene/butenes is a liquid-liquid extraction section in which said topped butadiene effluent feeds an intermediate zone of a first liquid-liquid extraction column, in which a stream of polar solvent, preferentially DMSO, is fed at the top. A saturated hydrocarbon solvent, preferentially pentane or cyclohexane, is fed at the bottom. The flow rates and also the ratio of the flow rates of polar solvent to hydrocarbon solvent are adjusted so that most of the butenes will be entrained by the hydrocarbon solvent and most of the butadiene will be entrained by the polar solvent.

The butenes/hydrocarbon mixture obtained at the top of the first extraction column is then treated in a first distillation column in order to obtain the butenes effluent at the top and the hydrocarbon solvent, which can be recycled, at the bottom.

The butadiene/polar solvent mixture then feeds the top of a second liquid-liquid extraction column in which the butadiene is extracted from the polar solvent by being brought into direct contact with a greater amount of hydrocarbon solvent than in the first liquid-liquid extraction column, which is introduced at the bottom of said second liquid-liquid extraction column.

The butadiene/hydrocarbon mixture obtained at the top of the second liquid-liquid extraction column is then treated in a distillation column in order to obtain the purified butadiene effluent at the top and the hydrocarbon solvent, which can be recycled, at the bottom.

Preferably, the liquid-liquid extraction columns of said section for separating butadiene/butenes are operated at a pressure of between 0.1 MPa and 1 MPa; and at a temperature of between 20 °C and 60 °C.

In another embodiment of the invention, said step b4″) comprises at least a distillation and an extractive distillation. The distillation step can be carried out upstream or downstream of the extractive distillation step. The extractive distillation can be carried out, in a non-limiting manner, with a solvent such as N-methylpyrrolidone, dimethylformamide or acetonitrile.

### Step b5) of Treating the Effluents

In accordance with one embodiment according to the invention, step b5) of treating the effluents is fed at least with the water/ethanol/acetaldehyde raffinate resulting from step b5′), and produces at least an ethanol-rich effluent, an acetaldehyde-rich effluent and a water-rich effluent. If the waste water effluent resulting from step b4′) or the alcoholic water effluent resulting from step b6) or the waste water effluent resulting from step b3′) have not undergone step b5′) of removing impurities and brown oils, they can directly feed step b5) of treating the effluents. Section b5) is also advantageously fed with a fraction of the ethanol feedstock.

Preferably, and unlike the prior art, no withdrawal is carried out with a loss of ethanol or acetaldehyde.

Preferably, said step b5) comprises at least two distillation sections: one “water and ethanol” distillation section and one “acetaldehyde” distillation section.

Said water/ethanol/acetaldehyde effluent resulting from step b5′) and optionally the waste water effluent resulting from step b4′) feed said acetaldehyde distillation section, in which acetaldehyde is separated so as to form an acetaldehyde-rich effluent, the residue from said acetaldehyde distillation section feeding a water and ethanol distillation section, making it possible to separate off an ethanol-rich effluent at the top and a water-rich effluent at the bottom. Since the alcoholic water effluent resulting from step b6) and the waste water effluent resulting from step b3′) do not contain acetaldehyde, they can directly feed said water and ethanol distillation section.

The ethanol-rich effluent resulting from step b5) predominantly consists of ethanol. “Predominantly” means more than 80% by weight, preferably more than 84% by weight. In a non-limiting manner, the ethanol-rich effluent resulting from step b5) may contain impurities such as water, ethyl acetate, butanol and hexanol.

The impurities other than water represent less than 10%, in a favored manner less than 5% and more preferentially less than 2% by weight of the stream.

The acetaldehyde-rich effluent resulting from step b5) predominantly consists of acetaldehyde and ethanol. “Predominantly” means more than 80% by weight, preferably more than 85% by weight. In a non-limiting manner, the acetaldehyde-rich effluent resulting from step b5) may contain impurities such as water, ethyl acetate or acetone.

The impurities other than water represent less than 10% and preferentially less than 5% by weight of the stream.

Said acetaldehyde-rich, ethanol-rich and water-rich effluents are then recycled to the rest of the process according to the invention. The fraction of said ethanol-rich effluent feeding step b1) is preferably at least 0.7, preferably at least 0.75, more preferably at least 0.8. The fraction of said water-rich effluent feeding said step b6) is advantageously between 0 and 0.3, very advantageously between 0 and 0.1, more advantageously between 0 and 0.01. The fraction of said water-rich effluent feeding said step b5′) of eliminating impurities and brown oils is advantageously between 0 and 1, preferentially between 0.3 and 0.6, and advantageously between 0.4 and 0.5.

In another embodiment of the invention, said acetaldehyde-rich, ethanol-rich and water-rich effluents undergo a purification step before being recycled to the rest of the process. “Purification” means placing said effluents in contact with adsorbents, for instance activated carbon, silica, alumina or else a functionalized polymeric resin. For example, an active carbon makes it possible to remove the traces of butanol and hexanol included in the ethanol-rich stream. For example, a basic resin makes it possible to remove the acetic acid present in the water-rich effluent. When the adsorbents are saturated and do not make it possible to ensure the purity of the acetaldehyde-rich, ethanol-rich and water-rich effluents, they are removed or regenerated for reuse.

Step b5′) of Removing Liquid Impurities and Brown Oils

In accordance with one embodiment according to the invention, a step b5′) of removing impurities and brown oils is fed at least with the ethanol/acetaldehyde/water effluent resulting from step b4) and with a fraction of the water effluent resulting from step b5), and produces at least an ethanol/acetaldehyde/water raffinate, a light brown oils effluent and a heavy brown oils effluent.

Preferably, said step b5′) comprises at least a washing/backwashing section, a light brown oil distillation section and a heavy brown oil distillation section.

Said preferential washing/backwashing section is fed at an intermediate point with said ethanol/acetaldehyde/water effluent resulting from step b4), advantageously mixed with the waste water effluent resulting from step b4′), the alcoholic water effluent resulting from step b6), and the waste water effluent resulting from step b3′), if the latter step is carried out, and preferably mixed with a fraction of the waste water effluent resulting from step b4′). Since these effluents are richer in water than the ethanol/acetaldehyde/water effluent resulting from step b4), their introduction as a mixture makes it possible to limit hydrocarbon losses in the raffinate.

Said preferential washing/backwashing section is fed at the bottom with a hydrocarbon effluent and at the top with a fraction of the water effluent resulting from step b5), which does not comprise ethanol or acetaldehyde. The hydrocarbon effluent and the fraction of the water effluent resulting from step b5) are fed at a temperature preferably of between 10 and 70 °C, preferentially between 45 °C and 55 °C. Said washing/backwashing section produces at the top a washing hydrocarbon extract loaded with a fraction of the impurities and of the brown oils, and at the bottom said ethanol/acetaldehyde/water raffinate.

Said washing/backwashing section is preferably operated at a pressure of between 0.1 MPa and 0.5 MPa, preferentially between 0.2 MPa and 0.4 MPa. Preferably, the addition of water to carry out the backwashing is such that the water content in the water/ethanol/acetaldehyde raffinate is greater than 30% by weight, preferably greater than 40% by weight.

In one embodiment, the contact between the two liquid phases in said washing/backwashing section takes place in a liquid-liquid extractor. Various contact forms can be envisaged. Mention may be made, in a non-limiting manner, of a packed column, a pulsed column, or an agitated partitioned column. In another embodiment, the contact between the two liquid phases in said washing/backwashing section takes place in a membrane contactor, or a cascade of membrane contactors. This contact method is particularly well suited to the system used. Specifically, water-ethanol-hydrocarbon mixtures are known to form stable emulsions, which may be problematic in a liquid-liquid extractor. The membrane contactor makes it possible to generate a substantial area of contact, promoting the transfer of the impurities and oils to the hydrocarbon phase, without generating an emulsion.

Said washing hydrocarbon extract feeds said light brown oil distillation section, which produces said light brown oil effluent as distillate, and a hydrocarbon residue comprising the heavy fraction of the brown oils.

Said light brown oil effluent is composed of impurities produced by reaction step b2), mainly diethyl ether, ethyl acetate and crotonaldehyde, and also of the light fraction of the brown oils, composed of impurities in smaller amounts, including pentene, isoprene, butanal and vinyl ethyl ether. This effluent may be burnt to provide some of the heat required for the hot oil circuit or for the steam boilers of the process, or distilled to recover a diethyl ether effluent and/or an ethyl acetate/crotonaldehyde effluent, which may either be upgraded, or recycled to the reaction section of step b2) to be reconverted.

Said hydrocarbon residue essentially contains the hydrocarbons serving for washing, but also the heaviest fraction of the brown oils. To avoid accumulation of the brown oils by recycling of the hydrocarbon effluent to the liquid-liquid extractor, a fraction of said hydrocarbon residue is treated in said heavy oil distillation section, consisting of a distillation column, which produces a hydrocarbon distillate essentially composed of hydrocarbons with a few remaining traces of brown oils and, as residue, said heavy brown oil effluent comprising more than 80%, preferentially more than 85%, of hydrocarbons and also the heaviest brown oils. The fraction of said hydrocarbon effluent sent to said oil distillation section is between 5 and 30% of the total flow rate of said hydrocarbon residue, and preferentially between 10 and 20%. The hydrocarbon distillate is mixed with the fraction of the hydrocarbon residue that has not been treated in said heavy oil distillation section, so as to form the hydrocarbon effluent feeding said washing/backwashing section.

This effluent, which preferably represents between 0.1 and 20% of the feedstock of said heavy oil distillation section, preferentially between 0.3 and 5%, may be burnt to provide some of the heat required for the hot oil circuit or the steam boilers of the process. A supply of hydrocarbons equivalent to the losses at the bottom of said heavy oil distillation section is necessary to keep the washing flow rate constant. This column is adjusted so as to keep the concentration of brown oils constant in the hydrocarbon recycling loop (hydrocarbon effluent/washing hydrocarbon effluent loop).

The light and heavy brown oil effluents are removed from the process.

The ethanol/acetaldehyde/water effluent resulting from step b4) mainly comprises ethanol, acetaldehyde and water, but also impurities such as diethyl ether, ethyl acetate and the brown oils as defined previously. These impurities may accumulate if they are sent back to reaction steps b1) and b2) in the acetaldehyde-rich distillation cut and/or the ethanol-rich distillation cut and if they are only partially converted in the reaction sections of steps b1) and b2). Step b5′) makes it possible to recover some of these impurities before step b5) of treating the effluents, making it possible to prevent demixing of the brown oils within the distillation columns, to simplify the distillation scheme, and to obtain, on conclusion of step b5), an ethanol effluent, an acetaldehyde effluent and a water effluent of greater purity than the prior art.

The washing of the ethanol/acetaldehyde/water effluent resulting from step b4) with a hydrocarbon effluent entrains certain impurities, while the backwashing of the hydrocarbon stream entrains some of the impurities and brown oils with a fraction of the water effluent resulting from step b5), so as to limit any loss of acetaldehyde and of ethanol.

Surprisingly, the applicant has discovered that it is possible to obtain liquid-liquid phase separation by adding certain hydrocarbons to the ethanol/acetaldehyde residue resulting from step b4). This result is surprising because the ethanol/acetaldehyde residue resulting from the step is very rich in ethanol and acetaldehyde, which are miscible with the hydrocarbons in any proportions. By suitable selection of the hydrocarbon, the applicant has discovered that it is possible to obtain liquid-liquid phase separation and thus to carry out liquid-liquid extraction to remove some of the impurities contained in the ethanol/acetaldehyde/water effluent resulting from step b4). Said hydrocarbon effluent may contain saturated and/or unsaturated and/or aromatic hydrocarbons, preferably saturated hydrocarbons. Said hydrocarbon effluent advantageously consists of a mixture of hydrocarbons having between 6 and 40 carbon atoms, preferably between 10 and 20 carbon atoms. In a non-limiting manner, said hydrocarbon effluent may be a desulfurized gas oil or kerosene fraction or alternatively a hydrocarbon fraction produced by a unit of Fischer-Tropsch type.

The addition of water to the washing/backwashing section enables better functioning of the process for removing the impurities and brown oils according to the invention.

Step b6) of Washing the Gaseous by-Products with Water

In accordance with one embodiment according to the invention, a step b6) of washing with water is fed with the gaseous by-products effluent resulting from step b4) and also with a fraction of the water-rich effluent resulting from said step b5) and produces at least one alcoholic water effluent.

The aim of said step b6) is to recover the small ethanol fraction entrained in said gaseous by-products effluent resulting from step b4), in order to improve the overall yield of the process.

The amount of water resulting from said step b5) required in said step b6) according to the invention is very low, unlike what is required in the prior art, because the vapor effluent resulting from step b2) was scrubbed in step b4) with an ethanol stream containing very little, or no, acetaldehyde. Only a small fraction of ethanol therefore remains in this flow, which can be easily recovered with a small amount of water compared to the amount of water that would have been required if there were traces of acetaldehyde in the gaseous by-products effluent resulting from step b4).

The water loaded with ethanol after washing is withdrawn from said step b6) and constitutes the alcoholic water effluent. It preferably feeds step b5) directly in the water-ethanol distillation section without encumbering the acetaldehyde distillation section. In another embodiment of the invention, it preferably feeds step b5′) of removing impurities and brown oils.

Step b) of the process according to the invention makes it possible to minimize the flow rate of effluents to be treated in the step of treating the effluents and to reduce butadiene losses as much as possible, making it possible to recover more than 98%, preferentially more than 99%, of the butadiene produced on conclusion of the reaction steps in said purified butadiene effluent.

### Third Reaction Section (Step c) of the Process According to the Invention)

The third reaction section 7 comprises at least one reactor into which the 1,3-butadiene 6 resulting from the second reaction section 5, the CO₂ 4 resulting from the first reaction section 2 and the hydrogen 9 from the reaction section 5 are introduced.

According to a preferred embodiment, step c) of the process according to the invention comprises the following substeps:
c1) a step of C—C coupling between two molecules of CO₂ and one molecule of 1,3-butadiene;
c2) a step of separating out hexenedioic acids;
c3) a step of reducing the hexenedioic acids formed in step c1) to give adipic acid.

### Step c1)

Advantageously, step c1) is carried out in at least one reactor in which the reagents are brought into contact with a metal precursor, a ligand and a reducing agent in a solvent. The reagents are the 1,3-butadiene resulting from the second reaction section and the CO₂ produced in the first reaction section.

The metal precursor is preferably a nickel (II) salt. More preferentially, the nickel salt is selected from the tetrabromonickelate salt of tetrabutylammonium (NiBr₄(DBA)₂), NiCl₂(DME), NiBr₂(DME).

The ligand is preferably a bidentate dinitrogen (N,N) ligand, such as bipyridine or phenanthroline derivatives. More preferentially, the ligand is selected from the ligands 2-methyl-4,7-diphenyl-1,10-phenanthroline, 4,7-diphenyl-1,10-phenanthroline and 6-methyl-2,2′-bipyridine.

The reducing agent is preferably a metal selected from zinc or manganese. The reducing agent is preferably manganese. It is also possible to carry out the reduction step using a metal-photochemistry or -electrochemistry pair.

The solvent is preferentially a polar aprotic solvent. The solvent advantageously has a dielectric constant at 20 °C of greater than 25.0 and/or a dipole moment of greater than 2.5 D. The solvent is preferably selected from N-methylpyrrolidone (NMP), N,N-dimethylformamide (DMF), N,N-dimethylacetamide (DMA), acetonitrile, dimethyl sulfoxide (DMSO), HMPT, hexamethylphosphoramide (HMPA), nitrobenzene, formamide, nitromethane, and propylene carbonate. Even more preferably, the solvent is N,N-dimethylacetamide (DMA).

Step c1) is advantageously carried out at a temperature of between 5 °C and 70 °C, preferably between 40 °C and 70 °C.

Before separation step c2), the reaction medium is preferably gradually neutralized with an acid such as HCl and with water.

### Step c2)

During separation step c2), the hexenedioic acids are advantageously extracted from the aqueous medium with ethyl acetate by means of liquid-liquid extraction. Preferably, the ethyl acetate effluent enriched in hexenedioic acids is dried then undergoes distillation. One of the effluents is mainly ethyl acetate, which can be recycled. Another effluent is enriched in hexenedioic acids which can be further purified by crystallization, for example, then diluted in a solvent such as methanol in order to feed the final step.

### Step c3)

Step c3) consists in reducing the hexenedioic acids to give adipic acid. Step c3) can be carried out in at least one reactor in which the hexenedioic acids, diluted in methanol, are introduced in the presence of a catalyst and a reducing agent.

The catalyst used in step c3) is preferably a metal precursor on a support or a metal alloy, such as palladium-on-activated carbon or Raney nickel. More preferentially, the catalyst is based on palladium supported on activated carbon.

Preferentially, the purified hydrogen originating from step b3) of the second reaction section can be used in step c3) as a reducing agent. Reduction step c3) is preferably carried out at a temperature of between 10 °C and 50 °C, and more preferentially between 15 °C and 25 °C.

### EXAMPLES

1/Case of the Synthesis of Adipic Acid from Glucose without Upgrading the CO₂ of the Fermentation Step

The equation for the glucose fermentation reaction is C₆H₁₂O₆→2C₂H₅OH+2CO₂. As a result, 66.7% of the carbon in a glucose molecule is converted to ethanol and 33.3% to CO₂.

For a feedstock of 170 Kta of glucose, fermentation would theoretically enable the production of 86.8 Kta of ethanol and 83 Kta of CO₂.

Assuming that the yield by weight of the conversion of ethanol to butadiene is 40%, it is possible to produce 34.7 Kta of butadiene, i.e. at best 93.9 Kta of adipic acid. If the CO₂ is not upgraded to adipic acid, up to 45.4% of the carbon from the glucose can be upgraded to adipic acid (cf. table 1).

2/Case of the Synthesis of Adipic Acid from Glucose with Upgrading of the CO₂ of the Fermentation Step

The equation for the glucose fermentation reaction is C₆H₁₂O₆→2C₂H₅OH+2CO₂. As a result, 66.7% of the carbon in a glucose molecule is converted to ethanol and 33.3% to CO₂. For a feedstock of 170 Kta of glucose, fermentation would theoretically enable the production of 86.8 Kta of ethanol and 83 Kta of CO₂.

Assuming that the yield by weight of the conversion of ethanol to butadiene is 40%, it is possible to produce 34.7 Kta of butadiene, i.e. at best 93.9 Kta of adipic acid. 56.6 Kta of CO₂ is needed to produce 93.9 Kta of adipic acid. Fermentation thus produces enough CO₂ for the sequence to be self-sufficient in CO₂. If the CO₂ is upgraded to adipic acid, up to 68.1% of the carbon from the glucose can be upgraded to adipic acid (cf. table 1).

TABLE 1

| | | % C that can be | % C that can be |
|---|---|---|---|
| | | upgraded to adipic | upgraded to adipic acid |
| Biomass | | acid without upgrading | with upgrading of the |
| upgrading | Production in | the CO₂ in the 1st step | CO₂ in the 1st step |
| products | Kt/a | (not in accordance) | (in accordance) |
| Ethanol | 86.8 | 45.4% | 45.4% |
| CO₂ | 83.0 | | 22.7% |
| Total | | 45.4% | 68.1% |

1. A process for converting biomass into adipic acid, successively comprising:
a) a step of treating the biomass to produce ethanol and CO₂;
b) a step of converting the ethanol obtained on conclusion of step a), to obtain 1,3-butadiene and dihydrogen; and
c) a step of synthesizing adipic acid from the 1,3-butadiene and dihydrogen obtained on conclusion of step b) and the CO₂ obtained on conclusion of step a).

2. The process as claimed in claim 1, wherein step a) comprises the following sub-steps:
a1) a step of pretreating the biomass to obtain a pretreated substrate;
a2) a step of enzymatic or chemical hydrolysis of the pretreated substrate obtained on conclusion of step a1) to obtain an enzymatic or chemical hydrolysis must; and
a3) a step of alcoholic fermentation of the enzymatic or chemical hydrolysis must obtained on conclusion of step a2) to obtain ethanol and CO₂.

3. The process as claimed in claim 2, wherein substep a1) is carried out by steam explosion under acid conditions at a temperature of between 150 °C and 250 °C and for a period of between 5 minutes and 30 minutes.

4. The process as claimed in claim 2, wherein substep a2) is carried out by enzymatic hydrolysis in the presence of Trichoderma reesei cellulases.

5. The process as claimed claim 1, wherein step b) comprises the following substeps:
b1) a step of converting the ethanol into acetaldehyde, comprising at least a reaction section fed at least with a fraction of the ethanol-rich effluent resulting from step b5), operated at a pressure of between 0.1 MPa and 1.0 MPa and at a temperature of between 200 °C and 500 °C in the presence of a catalyst, and a separation section making it possible to separate the effluent of said reaction section into at least a gaseous hydrogen effluent and a liquid ethanol/acetaldehyde effluent;
b2) a step of converting into butadiene, comprising at least a reaction section fed at least with a fraction of said ethanol/acetaldehyde effluent resulting from step b1), with a liquid ethanol-rich effluent resulting from step b3), with a fraction of the acetaldehyde-rich effluent resulting from step b5), operated in the presence of a catalyst at a temperature of between 300 °C and 400 °C and at a pressure of between 0.1 and 1.0 MPa, the feed flow rates being adjusted so that the ethanol/acetaldehyde molar ratio at the inlet of said reaction section is between 1 and 5, and a separation section making it possible to separate the effluent of said reaction section into at least a gaseous effluent and a liquid effluent;
b3) a step of treating the hydrogen, comprising at least a compression section compressing said hydrogen effluent resulting from step b1) to a pressure of between 0.1 MPa and 1.0 MPa and a gas/liquid scrubbing section fed at a temperature of between 15 °C and −30 °C with a fraction of said ethanol-rich effluent resulting from step b5) and with a fraction of said ethanol/acetaldehyde effluent resulting from step b1) and fed at a temperature of between 25 °C and 60 °C with said compressed hydrogen effluent, and producing at least a liquid ethanol-rich effluent and a purified hydrogen effluent;
b4) a step of extracting the butadiene, comprising at least a compression section compressing said gaseous effluent resulting from step b2) to a pressure of between 0.1 MPa and 1.0 MPa, a gas/liquid scrubbing section comprising a scrubbing column fed at the top, at a temperature of between 20 °C and −20 °C, with an ethanol stream consisting of said ethanol feedstock from the process and/or a fraction of the ethanol effluent resulting from step b5) and at the bottom with said gaseous effluent resulting from step b2), which is cooled, and a distillation section operated at a pressure of between 0.1 MPa and 1 MPa, fed at least with the liquid effluent resulting from said step b2) and with the liquid effluent from said gas/liquid scrubbing section, said step b4) producing at least one gaseous by-products effluent, a crude butadiene effluent and an ethanol/acetaldehyde/water effluent;
b4′) a step of first purification of the butadiene, comprising at least a gas/liquid scrubbing section fed at the bottom with the crude butadiene effluent resulting from b4) and at the top with a stream of water which may be a stream of water which originates outside said process for producing butadiene and/or a fraction of the water effluent resulting from step b5), said scrubbing section producing a pre-purified butadiene effluent at the top and a waste water effluent at the bottom;
b4″) a subsequent step of purifying the butadiene, which is fed at least with said pre-purified butadiene effluent resulting from said step b4′) and producing at least a purified butadiene effluent;
b5) a step of treating the effluents, which is fed at least with the water/ethanol/acetaldehyde raffinate resulting from step b5′), and producing at least an ethanol-rich effluent, an acetaldehyde-rich effluent and a water-rich effluent;
b5′) a step of removing impurities and brown oils, which is fed at least with the ethanol/acetaldehyde/water effluent resulting from step b4) and with the water-rich effluent resulting from step b5), and producing at least a water/ethanol/acetaldehyde raffinate, a light brown oils effluent and a heavy brown oils effluent; and
b6) a step of washing with water, which is fed with the gaseous by-products effluent resulting from step b4) and also with a fraction of the water-rich effluent resulting from said step b5), producing at least one alcoholic water effluent.

6. The process as claimed in claim 1, wherein step c) comprises the following substeps:
c1) a step of C—C coupling between two molecules of CO₂ and one molecule of 1,3-butadiene;
c2) a step of separating out hexenedioic acids; and
c3) a step of reducing the hexenedioic acids formed in step c1) to give adipic acid.

7. The process as claimed in claim 6, wherein substep c1) is carried out in the presence of a metal precursor, a ligand and a reducing agent in a solvent, wherein:
the metal precursor is a nickel (II) salt;
the ligand is a bidentate dinitrogen ligand;
the reducing agent is a metal selected from zinc or manganese; and
the solvent is a polar aprotic solvent.

8. The process as claimed in claim 6, wherein substep c1) is carried out at a temperature of between 5 °C and 70 °C.

9. The process as claimed in claim 6, wherein substep c3) is carried out in the presence of a catalyst and a reducing agent, said catalyst being palladium on activated carbon, and the reducing agent being the purified hydrogen originating from substep b3).

10. The process as claimed in claim 1, characterized in that the biomass is a lignocellulosic biomass.

11. A plant suitable for implementing the process as claimed in claim 1, said plant comprising:
a first reaction section (2) making it possible to produce ethanol (3) and CO₂ (4) from biomass (1);
a second reaction section (5) for converting the ethanol (3) into 1,3-butadiene (6) and dihydrogen (9); and
a third reaction section (7) making it possible to produce adipic acid (8) from 1,3-butadiene (6), CO₂ (4) and dihydrogen (9).
